(19) United States
(12) Patent Application Publication
(10) Pub. No.: US 2011/0004242 A1
(43) Pub. Date: Jan. 6, 2011

(54) KNOTLESS SUTURE FIXATION DEVICE AND METHOD

(76) Inventor: Robert P. Stchur, Punta Gorda, FL (US)

Correspondence Address: EDWARD M. LIVINGSTON, PA, 963 TRAIL TERRACE DRIVE, NAPLES, FL, 34103, US

(21) Appl. No.: 12/496,073

(22) Filed: Jul. 1, 2009

(51) Int. Cl. A61B 17/04

(52) U.S. Cl. 606/232

(57) ABSTRACT

A knotless suture fixation device (1) and method having a anchor (2) with a top portion (4), a middle portion (5) and a bottom portion (6). The top portion having at least two wings (7) and the bottom portion having an aperture (8) that passes through the anchor. The interior of the middle portion is threaded to accept a screw (3). The screw pushes against the wings to spread the wings outward as the screw is tightened. An opening (29) between the middle portion of the anchor and the bottom portion of the anchor allows the screw to pass through the aperture and make contact with a tip (10) of the anchor as the screw is tightened, thereby locking any sutures that have been inserted through the aperture in place. In an alternative embodiment, the anchor may have one or more press fit ridges (31) instead of wings.

This invention relates to suture fixation devices, more particularly, a knotless suture fixation device and method that permits a user to quickly and easily fixate a suture to a bone without the need to tie conventional suture knots.

Currently, during surgeries to repair torn tendons, such as arthroscopic rotator cuff operations, a tendon is positioned on and re-attached to the bone using a plurality of sutures which are inserted through the tendon, usually on the greater tuberosity, wherein the sutures are ultimately knotted to maintain tension on the tendon, thereby permitting a person to have function of the tendon once again. However, the act of tying knots and overall suture management is very time-consuming. In addition, the greater the number of sutures used, the more confusion is created when ultimately tying the suture knots.

There are currently devices on the market that are designed to fixate the tendon without having to tie suture knots. However, the current devices are complicated, unreliable and, like all current techniques, often require multiple implants if multiple sutures are being used. Sutures are commonly attached to these anchors by passing the suture through an eyelet or aperture located in the tip of an anchor prior to the anchor being mounted in a bone. These sutures are not tied or locked into the anchors leaving the possibility that they can become loose over time. Further, the anchors themselves may become loose over time due to their structure. In addition, the tension applied to the tendon is often unevenly distributed across the tendon because the anchors cannot hold multiple sutures.

Therefore, a need exists for a knotless suture fixation device and method that provides a strong anchor in a bone and can securely lock multiple sutures in place without the need to tie conventional suture knots.

The relevant prior art includes the following references:

| Pat. No. | | |
|---|---|---|
| (U.S. unless stated otherwise) | Inventor | Issue/Publication Date |
| 2008/0319478 | Foerester et al. | Dec. 25, 2008 |
| 7,309,346 | Martinek | Dec. 18, 2007 |
| 2007/0005068 | Sklar | Jan. 04, 2007 |
| 7,156,864 | Linter | Jan. 02, 2007 |
| 7,144,415 | Del Rio et al. | Dec. 05, 2006 |
| 2006/0235413 | Denham et al. | Oct. 19, 2006 |
| 7,090,690 | Foerester et al. | Aug. 15, 2006 |
| 7,083,638 | Foerster | Aug. 01, 2006 |
| 7,081,126 | McDevitt et al. | Jul. 25, 2006 |
| 2006/0161159 | Dreyfuss et al. | Jul. 20, 2006 |
| 2006/0116719 | Martinek | Jun. 01, 2006 |
| 6,916,333 | Schmieding et al. | Jul. 12, 2005 |
| 2005/0149122 | McDevitt et al. | Jul. 07, 2005 |
| 2005/0080455 | Schmieding et al. | Apr. 14, 2005 |
| 2005/0075668 | Lizardi | Apr. 07, 2005 |
| 2004/0243180 | Donnelly et al. | Dec. 02, 2004 |
| 6,780,198 | Gregoire et al. | Aug. 24, 2004 |
| 6,773,460 | Jackson | Aug. 10, 2004 |
| 6,652,561 | Tran | Nov. 25, 2003 |
| 6,641,597 | Burkhart et al. | Nov. 04, 2003 |
| 6,527,795 | Lizardi | Mar. 04, 2003 |
| 6,214,031 | Schmieding et al. | Apr. 10, 2001 |
| 6,117,162 | Schmieding et al. | Sep. 12, 2000 |
| 6,086,608 | Ek et al. | Jul. 11, 2000 |
| 6,027,523 | Schmieding et al. | Feb. 22, 2000 |
| 5,957,953 | DiPoto et al. | Sep. 28, 1999 |
| 5,851,219 | Goble et al. | Dec. 22, 1998 |
| 5,824,011 | Stone et al. | Oct. 20, 1998 |
| 5,814,071 | McDevitt et al. | Sep. 29, 1998 |
| 5,707,395 | Li | Jan. 13, 1998 |
| 5,236,445 | Hayhurst et al. | Aug. 17, 1993 |
| 5,156,616 | Meadows et al. | Oct. 20, 1992 |
| 4,632,100 | Somers et al. | Dec. 30, 1986 |

### SUMMARY OF THE INVENTION

The primary object of the present invention is to provide a knotless suture fixation device and method that does not require a surgeon to tie any sutures.

A further object of the present invention is to provide a knotless suture fixation device and method that will allow a surgeon to easily adjust the amount of tension on a suture and tendon.

An even further object of the present invention is to provide a knotless suture fixation device and method that will allow a surgeon to easily mount an anchor into a bone and lock a suture into place in one step.

A further object of the present invention is to provide a knotless suture fixation device and method that permits a surgeon to secure multiple sutures to a bone using only one anchor.

Another object of the present invention is to provide a knotless suture fixation device and method that reduces the amount of time the wound is exposed to the air, thereby reducing the risk of contamination and infection.

Another object of the present invention is to provide a knotless suture fixation device and method that reduces the length of time a person is spent under anesthesia.

The present invention fulfills the above and other objects by providing a knotless suture fixation device and method having a anchor with a top portion, a middle portion and a bottom portion, the top portion having at least two wings and the bottom portion having an aperture that passes through the anchor. The interior of the middle portion is threaded to accept a screw. The anchor may also have stress points that allow the wings to bend outward more easily while a screw is being inserted into the anchor. The screw comprises a head, a body and a tip. The head may be flat, but it is preferably tapered so that the head will exert pressure on the wings while being screwed downward into the threaded middle portion of the anchor, thereby bending the wings outward. Alternatively, the anchor may be secured without wings by the use of pressure fit ridges. In addition, the head may have ridges or teeth that lock the screw into place and prevent the screw from turning counter-clockwise, thereby preventing the screw from loosening after being fully engaged in a anchor. An opening between the middle portion of the anchor and the bottom portion of the anchor allows the screw to pass through the aperture and make contact with the tip of the anchor as the screw is tightened, thereby locking any sutures that have been inserted through the aperture in place. The tip of the screw is preferably flat but may have a raised edged, which maximizes the compression between the screw tip and the tip of the anchor, thereby ensuring a strong hold on the suture. Alternatively, the tip of the screw may have a conical shape so that the tip of the screw pierces through suture, thereby ensuring a strong hold.

To use the knotless suture fixation device, a suture is first passed through a torn tendon. Then, a hole is made in a bone using a punch or a drill. Next, the suture is passed through the aperture in the anchor. Then, the anchor and the suture are inserted into the hole in the bone so that the top of the anchor sits below the surface of the bone. Next, tension is placed on the suture to pull the torn tendon into the desired position on the bone. Then, a screw in the anchor is turned thereby exerting outward pressure bending on the upper portion of the anchor and bending the wings outward. In addition, as the screw is tightened, the tip of the screw extends all the way through the anchor to the tip of the anchor, thereby clamping the suture in place.

In an alternative embodiment of the knotless suture fixation device the top portion of the anchor does not have wings, rather the anchor is held in a bone by at least one press fit ridge located on the outer surface of the anchor. A threaded inner surface of the middle portion of the anchor extends through the top portion and to the top of the anchor. An opening between the middle portion of the anchor and the bottom portion of the anchor allows the screw to pass through the aperture and make contact with the tip of the anchor as the screw is tightened, thereby locking any sutures that have been inserted through the aperture in place.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a front view of a screw partially engaged in an anchor of the present invention;

FIG. 2 is a front view of a screw fully engaged in an anchor of the present invention;

FIG. 3 is a cross section of an anchor of the present invention;

FIG. 4 is a perspective front view of a anchor of the present invention having multiple wings;

FIG. 5 is a perspective front view of a screw of the present invention;

FIG. 6 is a top view of a screw having locking teeth fully engaged in an anchor of the present invention;

FIG. 7 is a front plan view of an alternative embodiment of the present invention;

FIG. 8 is a flow chart showing a method for securing a torn tendon to a bone using a knotless suture fixation device;

FIG. 9 shows a torn tendon being secured to a bone using a knotless suture fixation device of the present invention;

FIG. 10 is a front view of an alternative embodiment of the knotless suture fixation device having pressure fit ridges.

### DESCRIPTION OF THE PREFERRED EMBODIMENTS

For purposes of describing the preferred embodiment, the terminology used in reference to the numbered components in the drawings is as follows:

| | |
|---|---|
| 1. | knotless suture fixation device |
| 2. | anchor |
| 3. | screw |
| 4. | top portion of anchor |
| 5. | middle portion of anchor |
| 6. | bottom portion of anchor |
| 7. | wings |
| 8. | aperture |
| 9. | suture |
| 10. | anchor tip |
| 11. | stress point |
| 12. | threads |
| 13. | head |
| 14. | body |
| 15. | screw tip |
| 16. | teeth |
| 17. | raised edge |
| 18. | engagement means |
| 19. | suture is passed through tendon |
| 20. | make a hole in the bone |
| 21. | pass suture through aperture |
| 22. | the anchor and suture are placed in the |
| | bone |
| 23. | tension is placed on the tendon and the |
| | suture |
| 24. | the screw is tightened |
| 25. | excess suture is trimmed |
| 26. | tendon |
| 27. | bone |
| 28. | insertion tool |
| 29. | opening |
| 30. | interior surface of an anchor |
| 31. | press fit ridge |
| 32. | exterior surface |

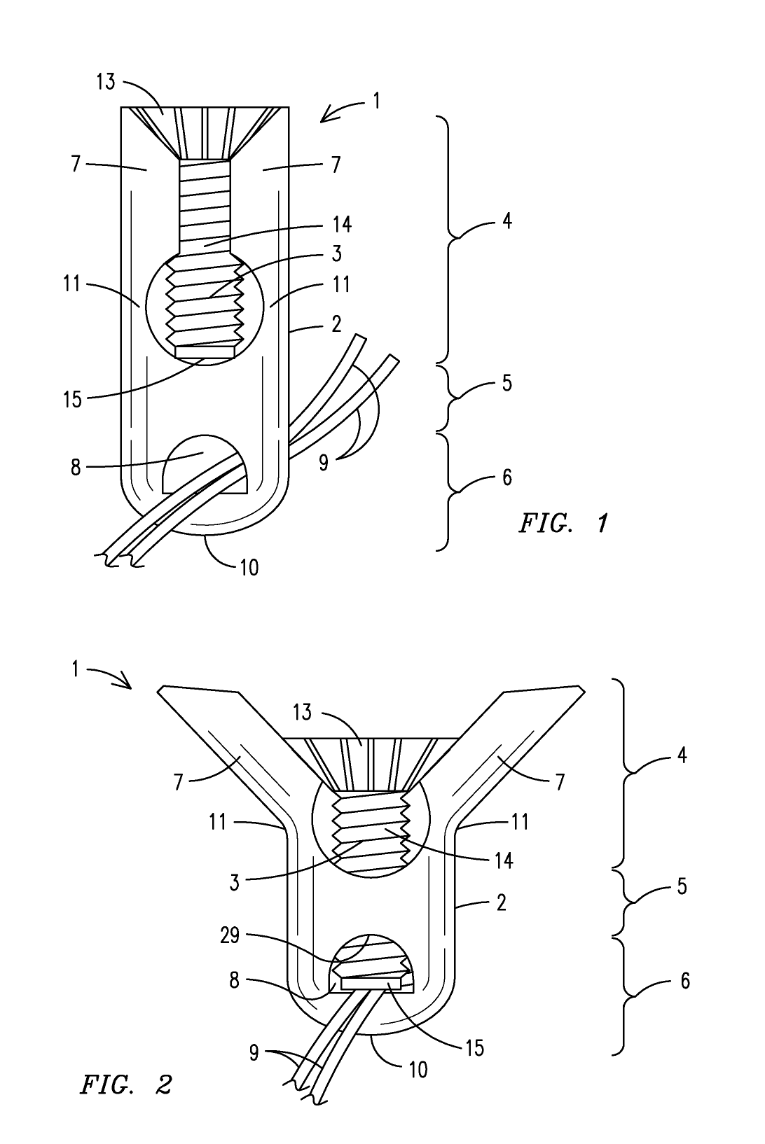

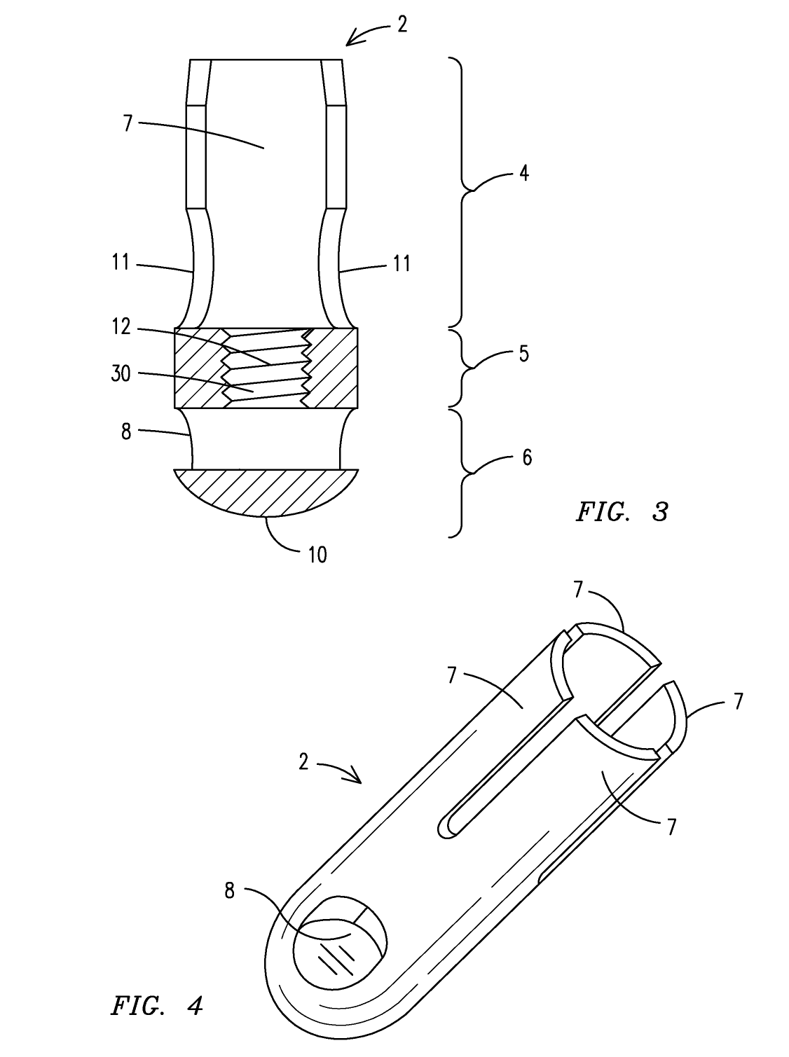

With reference to FIG. 1, a front view of a screw 3 partially engaged in an anchor 2 of a knotless suture fixation device 1 is shown. The anchor 2 comprises a top portion 4, a middle portion 5 and a bottom portion 6. The top portion 4 comprises at least two wings 7 and the bottom portion 6 comprises an aperture 8 through which a suture 9 may be passed. The wings 7 may have stress points 11 located at the bottom of the wings 7 to allow the wings 7 to bend outward more easily after a screw 3 has been fully engaged in the anchor 2, as shown further in FIG. 2. The screw 3 passes through the top portion 4 of the anchor 3 and engages the middle portion 5 of the anchor 2, which is threaded on the interior surface 30, as shown further in FIG. 3.

Now referring to FIG. 2, a front view of a screw 3 fully engaged in an anchor 2of the present invention is shown. The anchor 2 has a top portion 4, a middle portion 5 and a bottom portion 6. The top portion 4 has at least two wings 7 and the bottom portion 6 has an aperture 8 that passes through the anchor 2. The interior surface 30 of the middle portion 5 is threaded to accept the screw 3, as further shown in FIG. 3. The screw 3 comprises a head 13, a body 14 and a screw tip 15. The head 13 is preferably tapered so that the head 13 will exert pressure on the wings 7 while being screwed downward into the threaded middle portion 5 of the anchor 2, thereby bending the wings 7 outward and anchoring the anchor 2 in a bone. An opening 29 between the middle portion 5 and the bottom portion 6 of the anchor 2 allows the screw 3 to pass through the aperture 8 and make contact with a tip 10 of the anchor 2 as the screw 3 is tightened, thereby locking any sutures 9 that have been inserted through the aperture 8 of the anchor 2 in place.

Now referring to FIG. 3, a cross section of an anchor 2 of the present invention is shown. The anchor 2 comprises a top portion 4 a middle portion 5 and a bottom portion 6. The top portion 4 comprises at least two wings 7 and the bottom portion 6 comprises an aperture 8, through which a suture may be passed through, as shown in FIGS. 1 and 2, and a tip 10 which is preferably rounded. The wings 7 may have stress points 11 located on the bottom of the wings 7 to allow the wings 7 to bend outward more easily after the screw 3 has been fully engaged in the middle portion 5 of the anchor 3, which has threads 12 on the inside.

Now referring to FIG. 4, a perspective front view of an anchor 2 with aperture 8 of the present invention is shown having multiple wings 7 to secure the anchor 2 on all sides into a bone.

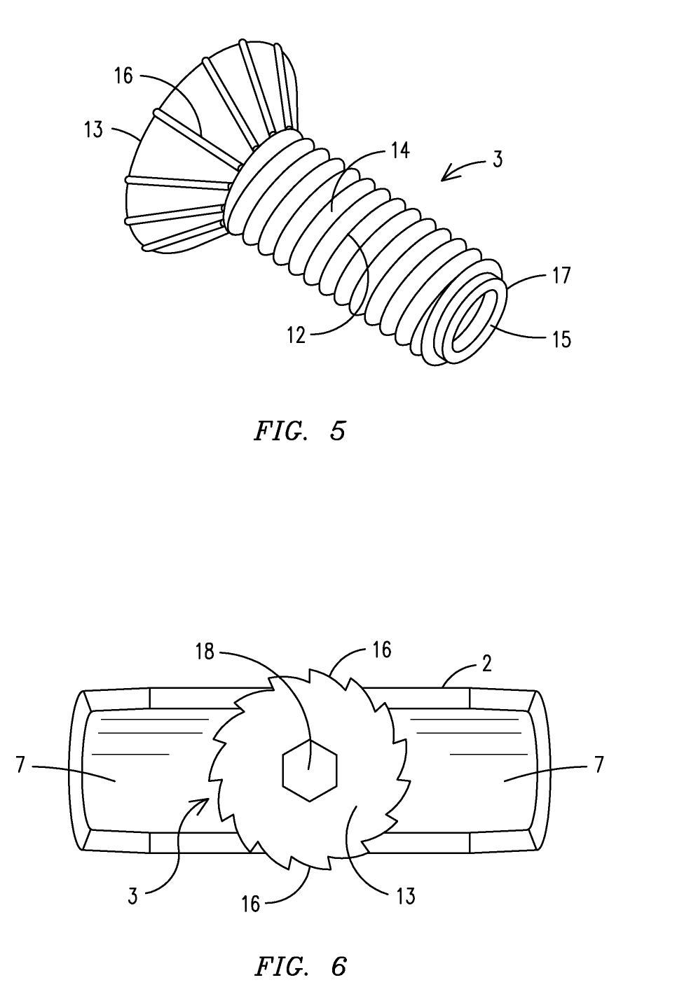

Now referring to FIG. 5, a perspective front view of a screw 3 of the present invention is shown. The screw 3 comprises a head 13, a body 14 and a tip 15. The head 13 is preferably tapered so that the head 13 will exert pressure on an upper portion of the anchor 2 while being screwed downward into the anchor 2, thereby bending the wings 7 outward, as shown in FIG. 2. In addition, the head 13 may have ridges or teeth 16 that secure the screw 3 in place in the anchor 2 preventing the screw 3 from turning counter-clockwise, thereby loosening the screw 3. The body 14 of the screw 3 has threads 12 so that the screw 3 will engage the threaded interior surface 30 of the anchor 2. The tip 15 of the screw is preferably flat but may have a raised edged 17, as shown here in FIG. 5. The raised edge 17 maximizes the compression on a suture 9 in the anchor 2, thereby ensuring a strong hold.

Now referring to FIG. 6, a top view of a screw 3 having locking teeth 16 fully engaged in an anchor 2 of the present invention is shown. The screw 3 has a head 13 with ridges or teeth 16 that lock the screw 3 into place in an anchor 2 to prevent the screw 3 from turning counter-clockwise becoming loose. In addition, the teeth 16 give a tactile feel, which aids a surgeon in preventing over tightening the screw 3 in an anchor 2. An engagement means 18, such as a projection, indentation, etc., is located on the of the screw head 13 head and is used in conjunction with an insertion tool to turn the screw.

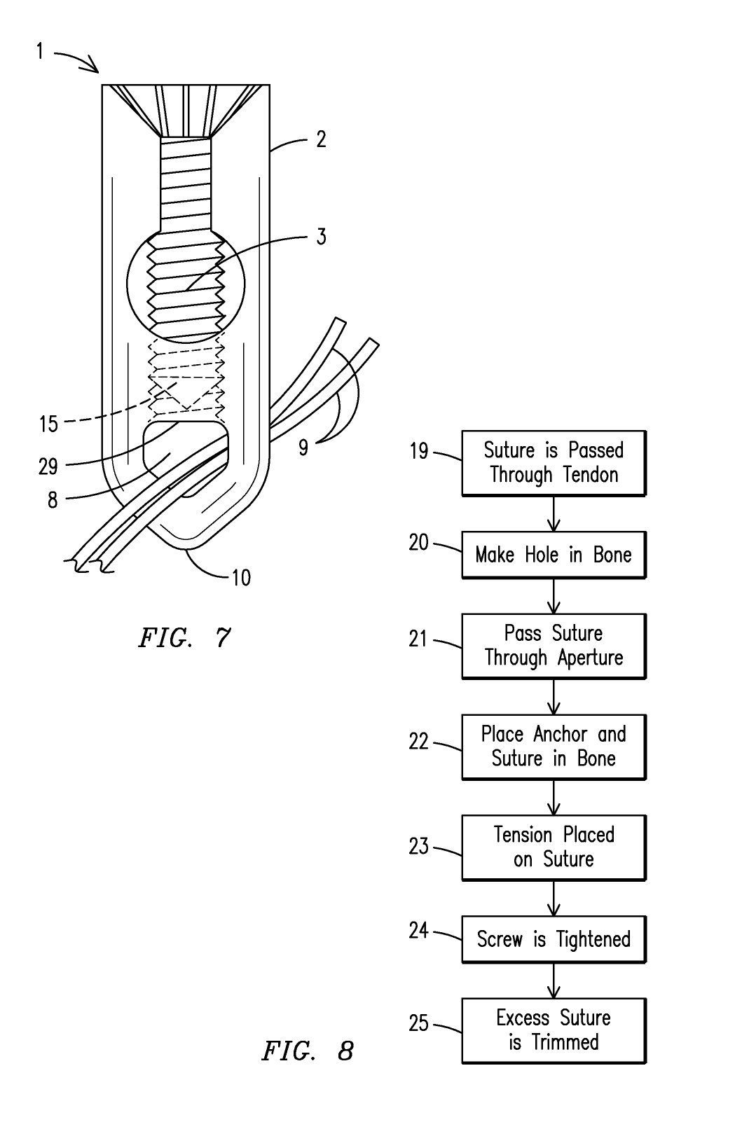

Now referring to FIG. 7, a front plan view of an alternative embodiment of a knotless suture fixation device 1 is shown. An opening 29 between a middle portion 5 and a bottom portion 6 of an anchor 2 allows a screw 3 to pass through an aperture 8 in the bottom portion of the anchor 2 and make contact with a tip 10 of the anchor 2 as the a screw 3 is tightened, thereby locking any sutures 9 that have been inserted through the aperture 8 in the anchor 2 in place. The aperture 8 and the tip 10 of the anchor 2 are angled to accept a conical shaped tip 15 of the screw 3. The conical shaped tip 15 of the screw 3 pierces through the suture 9 when the screw 3 is tightened, thereby locking the suture 9 in place.

Now referring to FIG. 8, a flow chart showing a method for securing a torn tendon to a bone using a knotless suture fixation device is shown. First, at least one suture is passed through a torn tendon 19. Then, a hole is made in the bone using a punch or a drill 20. Next, the at least one suture is passed through an aperture in an anchor 21. Then, the anchor and the suture are inserted into the hole in the bone so that the top of the anchor sits below the surface of the bone 22. Next, tension is placed on the suture to pull the torn tendon into the desired position on the bone 23. Then, a screw in the anchor is turned using an insertion tool thereby bending the wings of the anchor outward and securing the anchor in the bone 24. In additions as the screw is tightened, the tip of the screw extends all the way through the anchor to the tip of the anchor, thereby clamping the at least one suture in place. Finally, any excess of the at least one suture is trimmed 25.

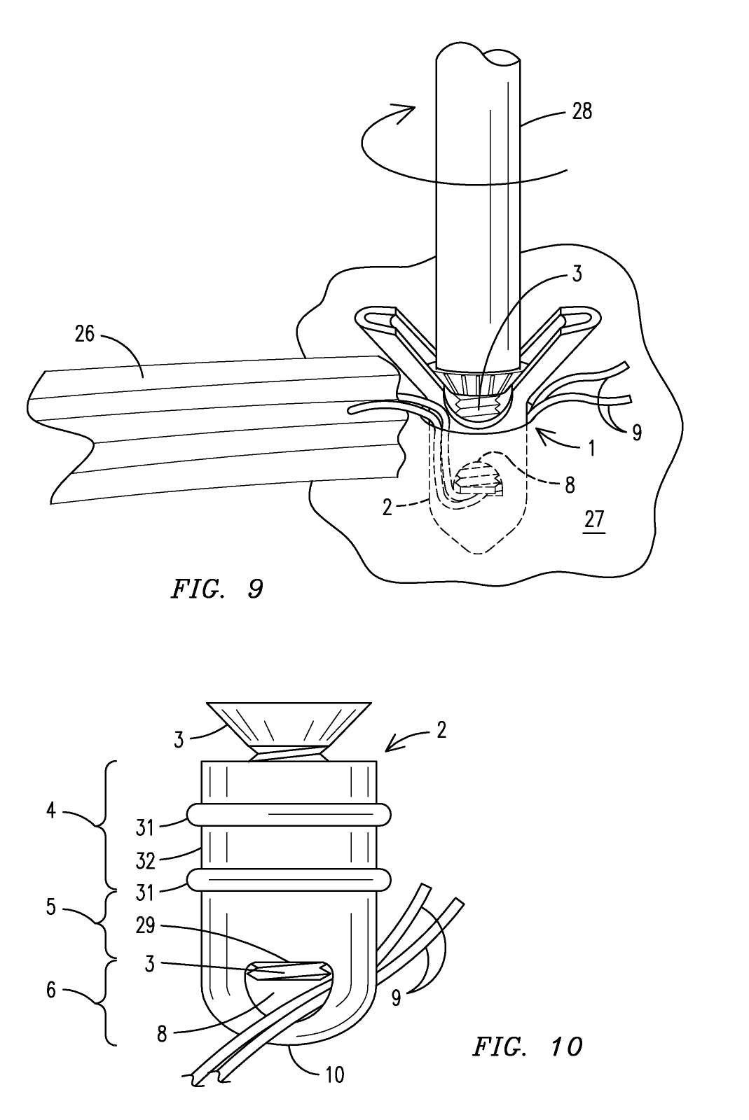

Now referring to FIG. 9, a torn tendon 26 being secured to a bone 27 using a knotless suture fixation device 1 of the present invention is shown. A suture 9 has been passed through a torn tendon 26 and then through an aperture 8 in an anchor 2. The anchor 2 and the suture 9 are then inserted into a hole in the bone 27 so that the top of the anchor 2 sits below the surface of the bone 27. Then, a screw 3 in the anchor 2 is turned using an insertion tool 28, thereby bending the wings 7 of the anchor 2 outward and securing the anchor 2 in the bone 27 and clamping the suture 9 in place.

Finally, referring to FIG. 10, a front view of an alternative embodiment of the knotless suture fixation device 1 having pressure fit ridges is shown. In the alternative embodiment, the top portion 4 of the anchor 2 does not have wings, rather the anchor 2 is held in a bone by at least one press fit ridge 31 located on the exterior surface 32 of the anchor 2. As the anchor 2 passes through the surface of a bone as the anchor is inserted into a hole in the bone, as described in FIG. 8, the at least one press fit ridge 31 compresses against the exterior surface 32 of the anchor 2. After the press fit ridge 31 passes through the surface of the bone, the press fit ridge extends away from the exterior surface 32 of the anchor 2, thereby securing the anchor 2 in the bone. A interior surface 30 of the anchor 2 has threads 12, which extend from the middle portion 5 of the anchor 2 through the top portion 4 of the anchor 2. An opening 29 between the middle portion 5 of the anchor 2 and the bottom portion 6 of the anchor 2 allows a screw 3 to pass through an aperture 8 and make contact with a tip 10 of the anchor 2 as the screw 3 is tightened, thereby locking sutures 9 that have been inserted through the aperture 8 in place.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A knotless suture fixation device comprising:
an anchor having a securing means, a threaded interior surface, a tip and an aperture through which at least one suture is passed;
a screw having a head, a tip and a threaded body which is inserted into the anchor, thereby engaging the threaded interior surface of the anchor; and
an opening between said threaded interior surface and aperture that allows the tip of the screw to pass through the aperture and make contact with the at least one suture and the tip of the anchors thereby locking said suture in the tip of the anchor.

2. The knotless suture fixation device of claim 1 wherein;
the securing means for the anchor comprises at least two wings.

3. The knotless suture fixation device of claim 2 further comprising:
stress points located at the bottom of said at least two wings.

5. The knotless suture fixation device of claim 1 wherein:
the head of said screw is tapered.

6. The knotless suture fixation device of claim 2 wherein:
the head of said screw is tapered.

7. The knotless suture fixation device of claim 1 wherein:
the head of said screw has at least one locking tooth.

8. The knotless suture fixation device of claim 2 wherein:
the head of said screw has at least one locking tooth.

9. The knotless suture fixation device of claim 5 wherein:
the head of said screw has at least one locking tooth.

10. The knotless suture fixation device of claim 6 wherein:
the head of said screw has at least one locking tooth.

11. The knotless suture fixation device of claim 1 wherein:
said tip of said screw has a raised edge.

12. The knotless suture fixation device of claim 2 wherein:
said tip of said screw has a raised edge.

13. The knotless suture fixation device of claim 1 wherein:
said tip of said screw is conical.

14. The knotless suture fixation device of claim 2 wherein:
said tip of said screw is conical.

15. A knotless suture fixation device comprising:
an anchor having a securing means, a threaded interior surface, a tip and an aperture through which at least one suture is passed;
a screw having a head, a tip and a threaded body which is inserted into the anchor, thereby engaging the threaded interior surface of the anchor; and
an opening between said threaded interior surface and aperture that allows the tip of the screw to pass through the aperture and make contact with the at least one suture and the tip of the anchor, thereby locking said suture in the tip of the anchor.

16. The knotless suture fixation device of claim 1 wherein:
the securing means for the anchor comprises at least one press fit ridge

17. The knotless suture fixation device of claim 15 wherein:
said tip of said screw has a raised edge.

18. The knotless suture fixation device of claim 15 wherein:
said tip of said screw is conical.

19. The knotless suture fixation device of claim 15 wherein:
the head of said screw has at least one locking tooth.

20. A method for securing atom tendon to a bone using a knotless suture fixation device comprising an anchor having a securing means, a threaded interior surface, a tip and an aperture through which at least one suture is passed, a screw having a head, a tip and a threaded body which is inserted into the anchor, thereby engaging the threaded interior surface of the anchor and an opening between said threaded interior surface and aperture that allows the tip of the screw to pass through the aperture and make contact with the at least one suture and the tip of the anchor, thereby locking said suture in the tip of the anchor comprising the steps of:
a. passing at least one suture having two ends through a torn tendon;
b. making a hole in a predetermined location on a bone;
c. passing the two ends of said at least one suture through an aperture in a anchor;
d. placing the anchor and the suture into the hole in the bone so that the top of the anchor sits below the surface of the bone; and
e. tightening a screw into the anchor thereby securing the at least one suture in the anchor and securing the anchor in the bone.

21. The method of claim 20 wherein step b further comprises:
using a punch to make the hole in a predetermined location on a bone.

22. The method of claim 20 wherein step b further comprises:
using a drill to make the hole in a predetermined location on a bone.

23. The method of claim 20 further comprising a step after step d of:
placing tension on the at least one suture to pull the torn tendon into a desired position on the bone.
